(19) United States
(12) Patent Application Publication
(10) Pub. No.: US 2010/0211081 A1
(43) Pub. Date: Aug. 19, 2010

(54) Microdrive and Modular Microdrive Assembly for Positioning Instruments in Animal Bodies

(75) Inventor: Darrell A. Henze, Collegeville, PA (US)

Correspondence Address: LAW OFFICES OF GRADY L. WHITE, LLC, 10605 Concord Street , SUITE 440, Kensington, MD, 20895, US

(73) Assignee: Merck Sharp & Dohme Corp., Rahway, NJ (US)

(21) Appl. No.: 12/718,747

(22) Filed: Mar. 5, 2010

Related U.S. Application Data
(63) Continuation-in-part of application No. PCT/US2008/075111, filed on Sep. 3, 2008.
(60) Provisional application No. 60/970,952, filed on Sep. 8, 2007.

(51) Int. Cl. A61B 19/00

(52) U.S. Cl. 606/130

(57) ABSTRACT

Microdrive and modular microdrive assembly for positioning the tips of substantially rigid medical and scientific instruments, such as electrodes, mechanical probes and needles, that are chronically implanted in animals, especially conscious and freely-moving animals, without passing the substantially rigid instruments through tubular guides or using immobilizing stereotactic surgical guide systems, such as arcuate rails mounted on relatively large and unwieldy external surgical headframes. Embodiments of the invention comprise a bottom plate adapted to be surgically attached to the animal and fixedly secured to a frame so that a void in the bottom plate is fixedly disposed over the implantation site. A carriage having a platform for mounting the substantially rigid instrument is alternately lowered and raised by rotating a drive rod connecting the carriage to the frame, enabling precise movement of the tip of the substantially rigid instrument into and out of the animal's body tissue.

### CROSS REFERENCE TO RELATED APPLICATIONS

This application is a Continuation-In-Part of International Application No. PCT/US2008/75111, filed on Sep. 3, 2008 (hereby incorporated by this reference), which claims the benefit of U.S. Provisional Application No. 60/970,952, filed on Sep. 8, 2007.

### FIELD OF ART

The present invention relates generally to devices and methods for positioning instruments, such as electrodes, mechanical probes and needles, in the body of an animal. More particularly, the invention provides a microdrive and a modular microdrive assembly for precisely positioning the tips of substantially-rigid medical and scientific instruments in animal bodies without passing the substantially rigid instruments through a tubular guide or mounting the instruments on large and unwieldy stereotactic surgical guide systems, which is particularly useful for chronic implantation of the instruments in conscious and freely-moving animals.

### RELATED ART

In the fields of medical and experimental neurophysiology, implantable signal recording electrodes (often referred to as sensors, electrical probes or simply “probes”) are carefully inserted into the brain tissue of patient and animal subjects via small passageways drilled into the subject's skull. When the probes have been implanted and precisely positioned in an area of the brain targeted for treatment or study, they can detect and record high-quality action potentials of nearby neuronal populations. Detecting, recording and analyzing such action potentials in the brains of humans and certain laboratory animals, such as monkeys, cats, rats and mice, for instance, permit doctors and scientific researchers to develop new and improved treatments for disorders, injuries and ailments affecting the human brain and/or nervous system, as well as improve their knowledge and understanding of brain activity in animals and humans. Implanted electrodes also are sometimes used, for example, to study and/or stimulate certain areas of the brain when the normal sensory pathways between the brain and other portions of the body have been damaged or destroyed due to traumatic injury or neurological disease. The information obtained from these procedures help engineers and technicians build better and more sophisticated neural prostheses for seriously injured human patients. The information also permits doctors and researchers to monitor and predict epileptic seizures or estimate the effects of anticipated brain surgery.

Signal recording instruments, such as neuronal probes, are usually driven into the target area of the brain using devices known as microdrives. Microdrives typically utilize one or more bent or angled carrier tubes, tubular support hoses or guide cannulae to hold, support and/or guide flexible wire electrodes as they are advanced into the brain tissue. For example, U.S. Pat. No. 5,928,143, issued to McNaughton, the disclosure of which is incorporated herein by reference, describes an implantable multi-electrode device in which the recording electrodes are slidably carried in an array of elongated guide cannulae having lower ends, which are parallel with and adjacent to each other, and upper ends that are inclined outwardly from a central vertical axis by an angle of thirty degrees. The outward thirty-degree incline of the guide cannulae provide sufficient spacing between adjacent cannulae so that the electrodes are capable of being independently adjusted.

Similarly, U.S. Pat. No. 5,413,103, issued to Eckhorn, the disclosure of which is also incorporated herein by reference, describes a microprobe and probe apparatus in which a plurality of microprobes are carried by a plurality of stretched elastic support hoses having lower ends that are parallel and adjacent to each other and upper ends that are inclined outwardly to provide sufficient spacing for a plurality of independently adjustable microdrives.

In “Semi-Chronic Motorized Microdrive and Control Algorithm for Autonomously Isolating and Maintaining Optimal Extracellular Action Potentials,” J. Neurophysiol. 93:570-579 (2005), authors Cham, Branchaud, Nenadic, Greger, Anderson and Burdick describe a motorized microdrive, comprising piezoelectric linear actuators capable of autonomously positioning four independent electrodes carried in hollow steel carrier tubes that each must be “pre-bent” by various amounts to accommodate placement in a common guide tube that is off-center to each carrier tube.

Because the known devices for driving instruments into animal bodies, including the devices described above, require mounting the instruments in carrier tubes, support hoses or cannula that are bent, inclined, angled or curved in some manner, they can only be used to drive and position very flexible (high ductility) instruments capable of sustaining large plastic deformations without damage or catastrophic fracture, such as electrodes made from wire. As compared to high ductility instruments, low ductility instruments, such as probes and electrodes made from silicon, carbon fiber, rigid metal, glass or hard plastic, are relatively stiff and brittle under shear stresses, and are, therefore, much more likely to snap, break or crack under the stresses that would be required to mount them in the bent, angled, curved or inclined guiding and support structures associated with many known instrument positioning devices. Accordingly, the known instrument positioning devices are recognized as unsuitable for use with substantially rigid low ductility instruments.

Nevertheless, substantially rigid low ductility instruments may be preferred, or even required, under certain circumstances, because they tend to resist kinking and bending better than high ductility instruments. As compared to flexible instruments, substantially rigid low ductility instruments can also be more effective because they are less susceptible to being deflected away from the targeted area by intervening tissue, protective membranes or other physiological structures or obstacles. Stiffer and stronger low ductility instruments significantly reduce the risk of encountering these problems because they are capable of withstanding higher compression and tension forces than high ductility instruments. Substantially rigid low ductility instruments also may be more easily extracted from the targeted area because they are less susceptible to catching, pulling, stretching or twisting while moving through tissue. Substantially rigid low ductility instruments may also provide better results than high ductility instruments due to some other inherent advantage or property, such as a higher degree of biocompatibility, better performance in a wider range of temperatures, or a higher resistance to corrosion or contamination due to the presence of water, heat, oxidizing agents and other chemicals, etc.

Stereotactic guide apparatuses capable of holding and positioning instruments that are not bent, angled, curved or inclined during mounting and surgical procedures have been introduced and used in the medical and scientific fields. But such apparatuses are typically mounted on and/or used in conjunction with external three-dimensional neurosurgical headframes or pulmonary surgery chestframes. Such headframes and chestframes are typically large and unwieldy devices that are bolted to the animal's body, which have swinging and/or rotating arcuate rails adapted to receive and hold the stereotactic guide apparatus at some precise distance away from the surface of the tissue or organ to be penetrated by the instrument. Consequently, using such stereotactic guide apparatuses to position instruments generally requires immobile and/or unconscious patients or animals, and thus have been found to be largely unsuitable and impractical for chronic implantation on conscious and freely moving subjects.

Conventional instrument positioning devices typically hold and extend the instruments through tubular-shaped guides and support structures, such as canulae, hoses or pipes. The primary purpose of these tubular-shaped guiding and support structures is to define the two-dimensional location above the tissue where the instruments will enter the tissue. However, rigid and substantially rigid instruments often have structures that are not uniform in diameter along their length, which makes it difficult, if not impossible, to place them in or move them in a precise manner through guiding and support tubes that characteristically have substantially uniform diameters along their length.

Accordingly, there is a need in the medical and scientific research fields for devices for driving and precisely positioning the tips of substantially rigid low ductility instruments, such as probes and needles made from silicon, carbon fiber, rigid metals, hard plastic, or other materials that have little or no elastic or plastic deformation ranges, without passing the substantially rigid instrument through a tubular guide and without requiring that the instrument be mounted on a relatively large and immobilizing frame required for a stereotactic guide apparatus. Such devices would be even more practical and convenient if they could also be used to drive and position the tips of flexible instruments (i.e., medium and high ductility instruments), such as wire probes and electrodes made from soft metals, like copper, silver, aluminum or gold.

### SUMMARY OF THE INVENTION

As will be described in more detail below, aspects and embodiments of the present invention address the above-described needs, as well as other deficiencies and problems associated with known devices, by providing a microdrive and microdrive assembly for positioning a wide range of different types of instruments, whether those instruments are flexible, inflexible or anywhere in between, but which are especially useful for positioning the tips of substantially rigid instruments having a given or fixed geometry. Stated generally, the microdrive comprises a frame, a bottom plate having a bottom void therein, the bottom plate being adapted to be fixedly secured to the frame and surgically attached to the body so that the bottom void is fixedly disposed about a location on the surface of the body where the tip is to be inserted, a drive rod rotatably mounted to the frame, and a carriage with a threaded bore and a platform for fixedly mounting the substantially rigid instrument, the platform being configured to hold the substantially rigid instrument so as to permit the tip to be extended through the bottom void toward the surface of the body without passing the substantially rigid instrument through a tubular guide. The drive rod has a threaded shaft, which passes through the threaded bore on the carriage so that the threads of the threaded shaft are in complementary contact with the threads of the threaded bore. Rotating the drive rod in one direction (e.g., clockwise) produces a force at the complementary contact that urges the platform on the carriage closer to the surface, thereby forcing the instrument held by the carriage to penetrate (or move further into) the body. Rotating the drive rod in the opposite direction (e.g., counterclockwise) produces an opposite force at the complementary contact that urges the platform on the carriage away from the surface, thereby retracting or extracting the instrument from the body.

When the instrument is an electrical device, such as an electrode, a flexible electric cable carries electrical signals between the instrument and an electrical connector mounted to the frame. The electrical connector is typically coupled to an interface cable that is coupled to a remote signal processor or remote signal generator. When the instrument is a fluid-transporting device, such as a needle, a flexible fluid tube carries fluids between the instrument and a fluid connector mounted to the frame, and the fluid connector is fluidly-coupled to a remote fluid reservoir. In another aspect of the present invention, there is provided a modular microdrive assembly for positioning instruments in the body of an animal, comprising a plurality of microdrives, as described above, which are secured to one or more plates (e.g., a top plate and a bottom plate) which serve to stabilize and orient the plurality of microdrives, and optionally, a protective cover that shields and protects the microdrives and associated connections from external forces.

The term “instrument” encompasses any tool, utensil or implement that is typically inserted and positioned in a body by applying forces which cause that tool, utensil or implement to pierce and penetrate the tissue of the body in a precise and controlled manner. Thus, the term “instrument” includes, without limitation, sensor and stimulation devices, such as electrical and mechanical probes, as well as fluid transporting devices, such as needles. The instruments may be made of any material, including, for example, silicon, carbon fiber, glass, metal, plastic or rubber, and may be fixedly mounted to the platform on the carriage using, for example, an epoxy adhesive, acrylic, polyurethane, cyanoacrylate, or any other type of reliable adhesive. The instrument may also be mounted to the platform on the carriage using a clamp, screw or clip.

Unlike conventional instrument positioning devices, the present invention can be used with substantially rigid, relatively unflexible low ductility instruments because it does not require that the instrument be bent, angled, twisted, stretched or otherwise subjected to elastic or plastic deformation for mounting purposes. Embodiments of the present invention also do not require using neurosurgical headframes, breast frames, chest frames, or any other type of large and immobilizing apparatus bolted to the body, which is designed to hold the positioning device away from the surface of the body where the tip of the instrument is to be inserted. Unlike the conventional systems, embodiments and variations of the present invention also achieve precise horizontal and vertical positioning within the animal body without requiring that the instruments be carried in or extended through guiding tubes, hoses or pipes.

Another aspect of the present invention provides a method for positioning the tip of a substantially rigid instrument in the body of an animal using the above-described microdrive, the method comprising the steps of: (1) fixedly mounting the substantially rigid instrument to the platform on the carriage; (2) surgically attaching the bottom plate to the body so that the bottom void is fixedly disposed about a location on the surface of the body where the tip is to be inserted; fixedly securing the frame to the bottom plate so that the tip of the substantially rigid instrument mounted on the platform extends through the bottom void toward the surface of the body without passsing the substantially rigid instrument through a tubular guide; and rotating the drive rod in one direction to produce a force at the complementary contact which urges the platform on the carriage closer to the surface, thereby forcing the tip of the substantially rigid instrument to penetrate the body.

### BRIEF DESCRIPTION OF THE DRAWINGS

The present invention and various aspects, features and advantages thereof are explained in detail below with reference to exemplary and therefore non-limiting embodiments and with the aid of the drawings, which constitute a part of this specification and include depictions of the exemplary embodiments. In these drawings:

FIG. 1 depicts an implanted microdrive assembly for positioning electrical instruments according to an embodiment of the present invention, the microdrive assembly comprising four independently-operable microdrives.

FIGS. 2A, 2B and 2C show, respectively, a left side orthogonal view, a right perspective view (from below) and a front perspective view (from above) of a microdrive for positioning electrical instruments according to an embodiment of the present invention.

FIGS. 3A, 3B and 3C show, respectively, a right perspective view (from above), a left perspective view (from below) and a left side orthogonal view of the frame for the microdrive.

FIGS. 4A through 4E show various views of five other components of the microdrive, including the carriage, the drive rod, the bracket, the electrical connector and flexible electric cable, all of which may be attached to the frame depicted in FIGS. 3A, 3B and 3C, in order to construct a microdrive according to an embodiment of the invention.

FIG. 5 shows an exploded view of the electrical instrument microdrive.

FIGS. 6A and 6B show a microdrive for positioning a fluid-transporting device, such as a needle, according to an alternative embodiment of the present invention. The fluid instrument microdrive is shown in the retracted position (FIG. 6A), as well as the extended position (FIG. 6B).

FIGS. 7A and 7B show, respectively, a top perspective view and a bottom perspective view of the bottom plate.

FIGS. 8A, 8B and 8C show left perspective views (from above and below) of a microdrive assembly for electrical instruments according to an embodiment of the invention.

FIGS. 9A and 9B show, respectively, a top perspective view and a bottom perspective view of the top plate for the microdrive assembly.

FIGS. 10A, 10B and 10C show, respectively, a left perspective view (from above), a left side orthogonal view and a bottom perspective view of a microdrive assembly for electrical instruments according to another embodiment of the invention.

FIGS. 11A, 11B and 11C show, respectively, a left perspective view (from above), a rear perspective view (from above) and a left perspective view (from below) of a protective cover for one embodiment of the invention.

FIGS. 12A and 12B show, respectively, a left perspective view (from below) and a right perspective view (from above) of the protective cover and interface cable connections.

FIG. 13 shows a microdrive assembly according to an alternative embodiment of the invention, the microdrive assembly being configured to hold and position two electrical instruments.

### DETAILED DESCRIPTION OF EXEMPLARY EMBODIMENTS

Prior to the present invention, it has not been possible to use substantially rigid, low ductility instruments, such as silicon or carbon fiber probes, glass and metal needles, in certain medical and scientific research applications because the substantially rigid instruments could not be dynamically altered or deformed (i.e., bent, angled or curved) in order to install them on the known instrument driving devices. Microdrives and modular microdrive assemblies according to embodiments of the present invention are capable of positioning both substantially rigid low ductility instruments and substantially flexible high ductility instruments in animal bodies, without altering their given geometries and without using a stereotactic surgery guiding apparatus.

Microdrive

As previously stated, the microdrive comprises a frame, a drive rod rotatably mounted to the frame, and a carriage having a platform configured to hold the instrument in a position adjacent to a location on the surface of the body where the instrument is to be inserted. A bottom plate having a bottom void therethrough is adapted to be fixedly secured to the frame and surgically attached to the body so that the bottom void is fixedly disposed about the location on the surface of the body where the tip is to be inserted. The drive rod has a helical threaded shaft, which passes through a complementary helical threaded bore on the carriage so that both the drive rod and the carriage are removably attached to the frame, and the threads of the threaded shaft are in complementary contact with the threads of the threaded bore. The instrument may or may not be in actual contact with the surface of the body prior to penetration. Turning the drive rod in one direction (whether by manual or automatic means) produces forces at the complementary contact that move the platform on the carriage down the shaft of the drive rod and closer to the surface of the body. This movement causes the tip of the instrument mounted to the platform on the carriage to penetrate the body, or move further into the body. Reversing the rotation of the drive rod produces opposite forces at the complementary contact which cause the platform on the carriage to move up the shaft of the drive rod and away from the surface of the body, thereby retracting or extracting the tip of the instrument.

The instrument may be a mechanical probe, an electrical device, such as an electrical probe, or a fluid transporting instrument, such as a needle. If the instrument is an electrical device, embodiments of the invention provide an electrical connector, which is mounted to the frame, and a flexible electrical cable (or “lead”), which carries electrical signals recorded by the instrument to the electrical connector. The electrical connector is typically coupled to an interface cable, which is configured to carry the electrical signals from the microdrive to a remote signal processor. The electrical connector and flexible electrical cable may also be configured to carry electrical signals in the opposite direction, so that electrical signals produced by a remote signal generator and carried by the interface cable to the electrical connector mounted to the frame may be transmitted to the instrument via the flexible electrical cable (and subsequently introduced into the body of the animal). In some cases, multiple electrical probes, or probes having multiple channels, will be mounted to the microdrive, in which case multiple flexible electrical cables, or flexible cables having multiple transmission channels, may be employed to carry multiple signals from the probe (or probes) to the electrical connector. Where multiple flexible cables are needed to accommodate multiple probes or multiple channels, a flexible ribbon cable, comprising multiple electrical leads, may be used. The interface between the electrical connector on the one hand, and the remote signal processor or generator on the other hand, may comprise an electrical conductor (e.g., a physical wire or cable) or a wireless transmitter/receiver configured to communicate using electromagnetic waves (e.g., radio).

If the instrument mounted to the carriage is a fluid transporting device, such as a needle, then embodiments of the invention may be utilized to precisely position the fluid transporting device in the animal body prior to injecting or collecting fluids. In this case, a fluid connector is coupled to a flexible tube that transports the fluids between the fluid connector and the instrument. The fluid connector is also connected via an interface tube to an external or remote fluid reservoir, pumping mechanism, or both. This arrangement permits fluids extracted from the body with the instrument to pass through the flexible tube, through the fluid connector and the interface tube, and into the remote fluid reservoir. The arrangement may also be used to move fluid in the opposite direction. The fluid connector, the flexible tube or the instrument, or all of them, may contain flow control valves that permit the fluid to travel in only one direction, thereby preventing backward flows and possible contamination of the source.

The carriage on the microdrive of the present invention includes at least one flange (preferably two) that are in slidable contact with the frame, which stabilizes the carriage against compression and tension forces and inhibits the carriage from rotating around the rotational axis of the drive rod. A bracket, coupled to the frame and in slidable contact with the carriage, further stabilizes the carriage, and inhibits the carriage from rotating about an axis substantially transverse to the rotational axis of the drive rod.

In some embodiments, the upper end of the drive rod passes entirely through and out of the top of the frame. In this case, the portion of drive rod extending out of the top of the frame may comprise or be attached to one of a variety of different structures to facilitate rotating the drive rod, such as a turnable knob, a crank, a thumbwheel, a bolt head, a slotted head, a headless slot, a socketed head, a headless socket, a Phillips head, a square-drive head, a Torx head, a Tri-Wing head, a Torq-Set head or a spanner head. However, automatic and/or machine-controlled rotation may be achieved by coupling to the drive rod any suitable motor drive mechanism, including, for example, a direct current (DC) motor, a stepper motor, a servo motor, or a piezoelectric motor. Such motor drive mechanisms, which are already known in the art, may be configured to automatically rotate the drive rod in very precise, predetermined increments, which causes the instrument attached to the carriage to move into or out of the tissue in very precise, predetermined increments. The lower end of the drive rod may be secured to the frame via any fastener or combination of fasteners that will not impede the drive rod's rotation, including, for example, a washer, a nut, a double nut, a c-clip, a pin, or the like.

Modular Microdrive Assembly

A modular microdrive assembly according to an embodiment of the invention comprises one or more microdrives secured to two plates (a top plate and a bottom plate). As previously stated, the bottom plate is adapted for surgical attachment to the region on the animal's body where the instrument is to be inserted. For example, if the instrument is to be inserted into the brain tissue of a laboratory mouse, then the bottom plate may be configured, in terms of its size and shape, for attachment to the mouse's skull immediately adjacent to the targeted brain tissue. The bottom plate has within it a bottom void (comprising, for instance, some type of hole, aperture, bore, slit, passageway, notch, cutout or other opening) that, after attachment to the animal's body, will be adjacent to a location on the surface of the body where the tip of the instrument is to be inserted. The bottom void is sufficiently large to permit the penetrating tip of the instrument to pass through it as the instrument moves toward or away from the targeted area.

The plurality of microdrives on the modular microdrive assembly have a plurality of drive rods. Rotating the plurality of drive rods in one direction (although not necessarily the same direction) produces forces at the complementary contacts that urge the carriages on the plurality of microdrives to move toward the surface of the body, thereby pushing a plurality of instruments mounted to the carriages through the bottom void and the tips of the instruments into the targeted area. When the direction of rotation on the drive rods is reversed, opposite forces are produced at the complementary contacts that urge the carriages away from the surface of the body, thereby pulling the tips of the instruments mounted to the carriages through the bottom void and out of the targeted area. The top plate and bottom plates, which combine to significantly increase the stability of the modular microdrive assembly while it is in use, may be secured to the microdrives using screws, adhesive, or any other suitable means of attachment. However, screws may be preferred because they typically permit the components of the microdrive assembly to be more easily assembled, disassembled and reused.

The plurality of drive rods may be configured to rotate independently. Thus, embodiments of the present invention may used to independently insert and position multiple instruments in the body of an animal (i.e., independent positioning of any subset of the multiplicity of instruments), and maintain the independent positions over an extended period of time. The ability to independently adjust the vertical displacement of a subset of the multiplicity of instruments coupled to the drive rods may be particularly useful, for instance, when the multiplicity of instruments includes instruments of different types (e.g., a mix of electrodes, mechanical probes and needles mounted on a single microdrive assembly concurrently). Alternatively, the plurality of drive rods may be configured to rotate in a synchronized manner in order to facilitate precisely positioning a plurality of instruments simultaneously at substantially the same depth in the animal body and maintaining that same vertical position over an extended period of time.

Exemplary Uses and Applications

It is anticipated that embodiments of the invention may be utilized in a variety of fields, including without limitation the field of experimental neurophysiology. Thus, embodiments of the invention may be surgically implanted on the skulls of laboratory animals, such as rats, mice, birds and monkeys, and used to record neuronal signals originating in the brain while the animal is conscious and mobile. It should be noted, however, that embodiments of the invention may be adapted for surgical implantation on other parts of animal bodies, such as the spinal cord or chest plate, for instance, and then used to precisely position instruments in other biological structures, including, for example, nerve tissue in the spinal cord, blood vessels, air passages, muscles or organs located within the chest cavity, and other parts of the body. Embodiments of the invention may also be used in research experiments or treatment procedures involving anesthetized and/or restrained animals.

Conscious and behaving laboratory animals that have microdrive assemblies implanted on their bodies may tend to tug and pull on the assembly or the interface cable attached to the assembly, which may cause damage to the assembly or disconnect the interface cable or interface connectors. To mitigate this problem, modular microdrive assemblies according to some embodiments of the invention include protective covers that at least partially enclose the interface cable and the microdrives in the microdrive assembly, thereby providing added protection against damage or disconnections resulting from such external forces. The protective cover comprises a substantially rigid receptacle having a hollow interior chamber into which the protected portions of the microdrives and interface cable extend. The interface cable may be clamped or screwed to the protective cover so that pulling and tugging forces introduced to the interface cable are far less likely to disconnect the interface cable from the microdrives. Instead, these forces are transmitted to and dissipated by the entire microdrive assembly, which is itself rigidly affixed to the animal's head or body. Preferably, but not necessarily, the top of the protective cover has holes in it that permit the drive rods to be rotated without disengaging the interface cable or removing the protective cover from the top plate.

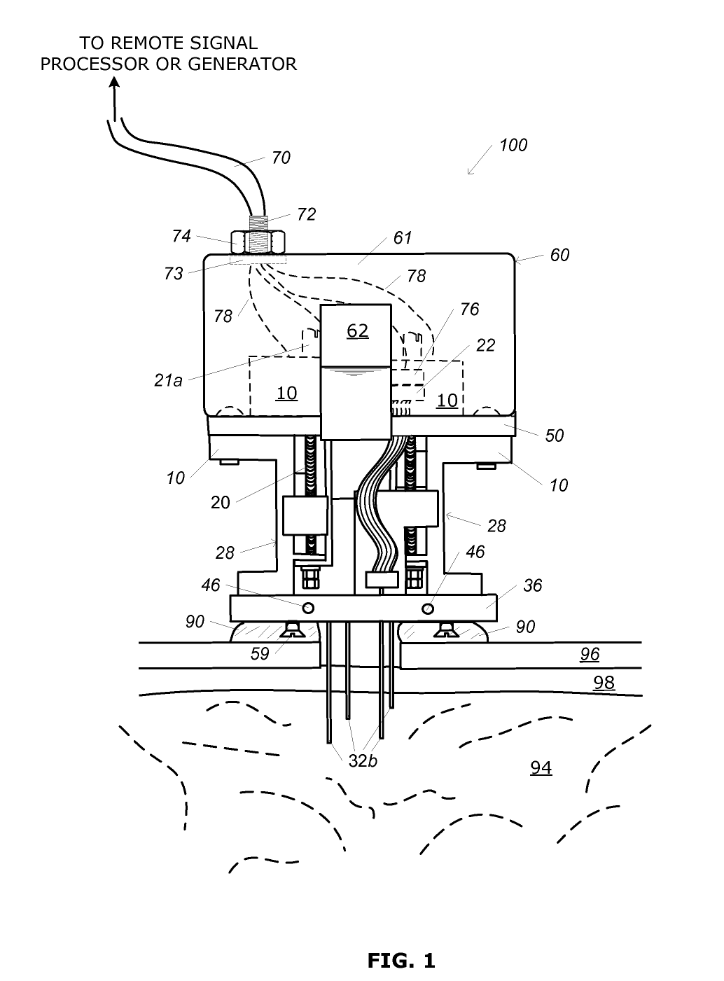

Exemplary microdrives and modular microdrive assemblies according to embodiments of the invention will now be described in more detail with reference to the figures. FIG. 1 shows a modular microdrive assembly 100 for positioning electrical instruments comprising four independently-operable electrical microdrives 28 for positioning electrical instruments. The electrical microdrives 28 are secured to a top plate 50 and a bottom plate 36. A protective cover 60 having a receptacle 61 is clipped to top plate 50 by two clips 62 located on opposite sides of protective cover 60 (only one of the clips is visible in FIG. 1). An interface cable 70, comprising a plurality of interface wires 78, is coupled to the top of protective cover 60 via interface coupling screw 72 and interface coupling nut 74. Interface wires 78, which pass into and through interface cable 70, are in electrical communication with one or more interface connectors 76 and one or more remote signal processors or signal generators (not shown). The one or more interface connectors 76 are electrically coupled to one or more electrical connectors 22, which are mounted to the sides of the frames 10 on the electrical microdrives 28.

Receptacle 61 has an inner hollow chamber that is sufficiently large to accommodate a flange 73 on the lower end of interface coupling screw 72, interface wires 78, interface connectors 76 and the portions of the electrical microdrives 28 that extend through and above top plate 50. The inner hollow chamber of receptacle 61 also protects slotted heads 21a fitted to the tops of drive rods 20, the upper portions of the frames 10 and the electrical connectors 22 of the electrical microdrives 28.

As shown in FIG. 1, modular microdrive assembly 100 is mounted on the skull 96 of an animal, such as a mouse or rat, so that the tips of four electrical instruments 32b (e.g., neuronal probes) attached to electrical microdrives 28 may be advanced through a passageway in the skull 96 and the dura 98 to penetrate the animal's brain tissue 94. In order to accomplish this, the bottom plate 36 is attached to the subject animal's skull 96 using dental cement 90 (or some other reliable epoxy or adhesive) placed between the bottom plate 36 and the animal's skull 96 during the implantation procedure. The bottom plate 36 is adapted to receive a plurality of inverted slope-headed anchoring screws 59, which are immersed in the dental cement 90. The sloping heads of the screws 59 lodge in the dental cement 90 and help secure the bottom plate 36 (and thus the entire microdrive assembly 100) to the skull 96. In addition to slope-headed screws, or as an alternative, metal pins (not shown in FIG. 1) may be inserted into pinholes 46 and inclined downward toward the skull to provide additional structure that can be screwed or cemented to the skull's surface.

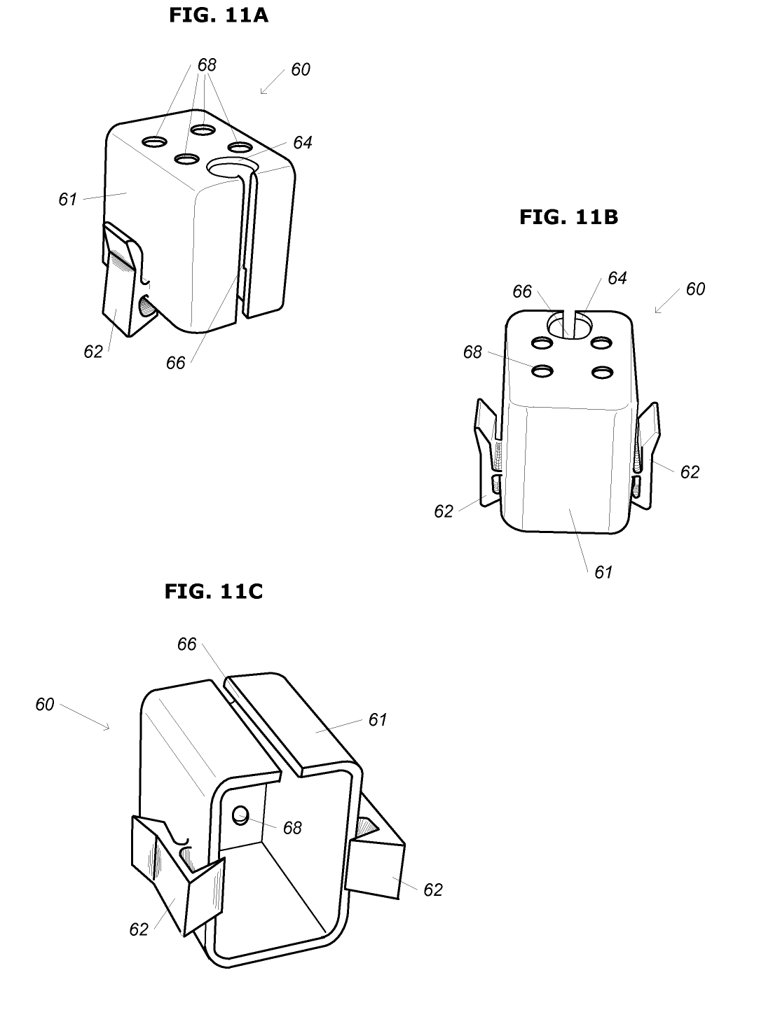

Typically, modular microdrive assembly 100 is mounted to the skull 96 so that, prior to rotating the drive rods 20, the penetrating tips of the electrical instruments 32b are close to the surface of the exposed dura 98 or the exposed brain tissue 94 where the instruments are to be inserted. The instruments may then be lowered into dura 98 and brain tissue 94 by, for example, inserting a screw driver through holes located in the top of protective cover 60 (best shown in FIGS. 11A-C) to engage and rotate the slotted heads 21a fitted to the tops of the drive rods 20.

Although the exemplary modular microdrive assembly 100 shown in FIG. 1 contains four electrical microdrives 28 and is shown mounted to an animal's skull 96, it should be apparent that simple modifications to the protective cover 60, the top plate 50 and the bottom plate 36 may be implemented in order to accommodate attaching fewer or more microdrives, as well as implantation sites on the animal's body other than the skull, such as the chest cavity, for instance. Thus, it will be appreciated that the exact geometry and dimensions of modular microdrive assemblies according to embodiments of the present invention may vary substantially depending on the number of microdrives in the assembly, the number of electrical instruments to be positioned, as well as the implantation site and its geometry. As previously stated, each one of the plurality of drive rods may be configured to rotate independently from the rotation of the other drive rods, thereby enabling independent vertical positioning of any one or more instruments in a multiplicity of instruments that may be connected to the microdrives concurrently. The independent rotation of drive rods also facilitates attaching and using a mix of different types of instruments (e.g., electrodes, mechanical probes and needles) on a single implanted microdrive assembly simultaneously.

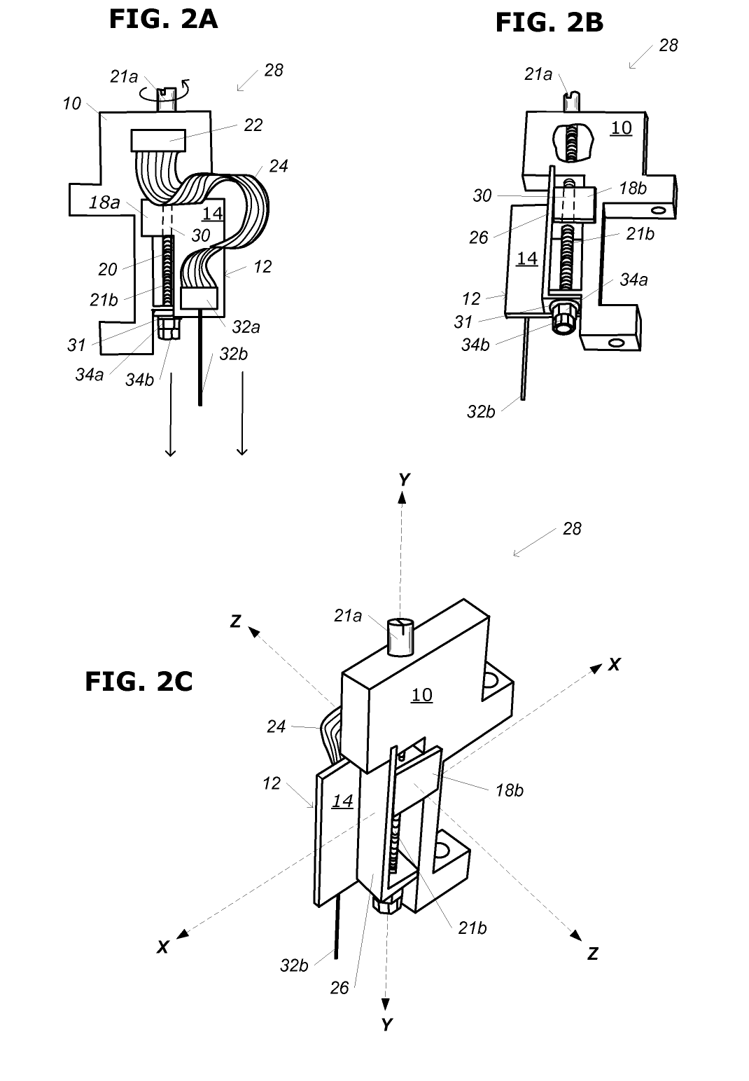

FIGS. 2A, 2B and 2C provide more detailed views of the electrical microdrives 28 incorporated into the exemplary module microdrive assembly shown in FIG. 1. FIG. 2A shows a left side orthogonal view of the electrical microdrive 28, while FIGS. 2B and 2C show, respectively, a right perspective view (from below) and a front perspective view (from above) of that same electrical microdrive. Superimposed on FIG. 2C are the X, Y and Z axes of an imaginary three-dimensional coordinate system having an origin located at the center of electrical microdrive 28.

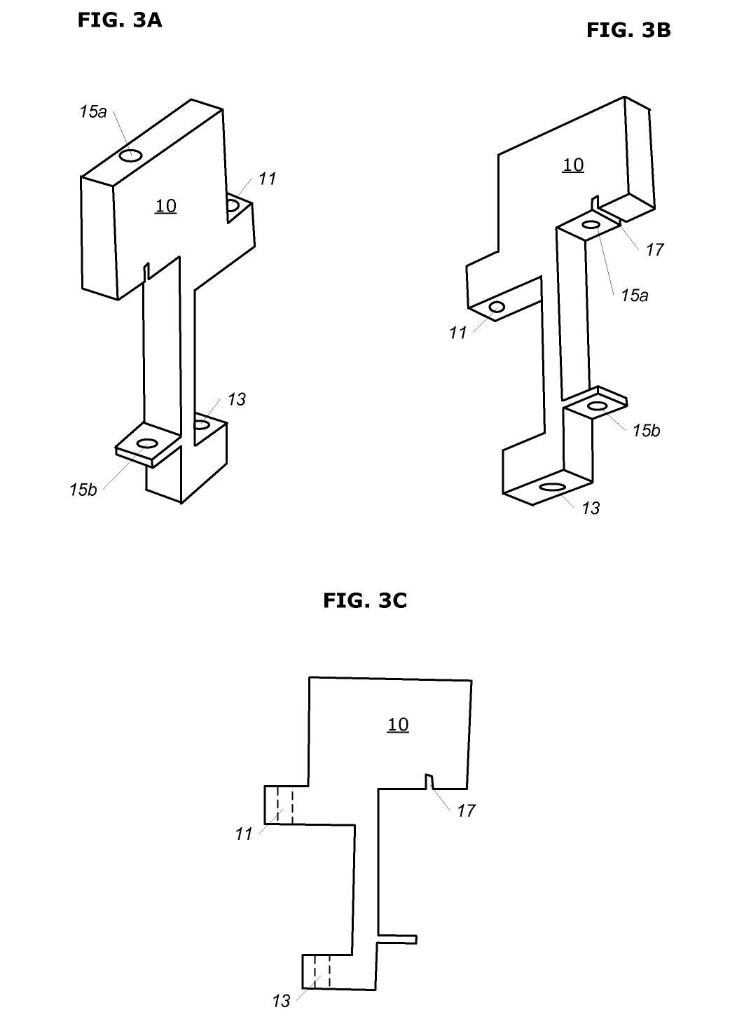

As shown in FIGS. 2A, 2B and 2C, the electrical microdrive 28 includes a frame 10, a carriage 12, a drive rod 20, a bracket 26, a flexible electrical cable 24 and an electrical connector 22. The upper end of drive rod 20 comprises a slotted head 21a, while the lower end comprises a threaded shaft 21b. The outer threads of the threaded shaft 21b are in complementary contact with the inner threads of threaded bore 16 on carriage 12. The location of the complementary contact is indicated in FIGS. 2A and 2B by the reference numeral 30. Electrical cable 24 is electrically coupled to the upper end 32a of the electrical instrument, which is mounted to a platform 14 on carriage 12. Rotating slotted head 21a rotates threaded shaft 21b, which creates forces at the complementary contact 30 that causes carriage 12 and electrical instrument 32a-b to move downward and into the animal body. Electrical microdrive 28 also includes a washer 31 and two nuts 34a and 34b, which secure the bracket 26 and drive rod 20 to the frame 10. Detailed descriptions of each of these components are provided below with reference to FIGS. 3A-3C, 4A-4E and 5.

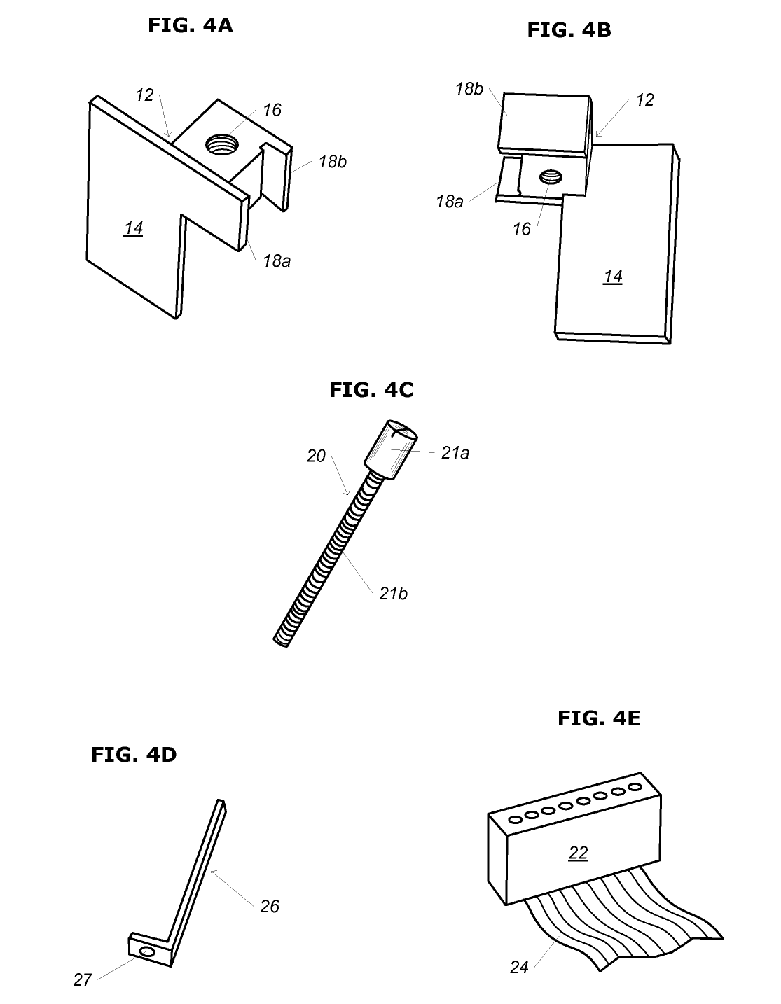

FIGS. 3A-3C provide more detailed views of the frame 10 incorporated into the electrical microdrive 28 shown in FIGS. 1, 2A, 2B and 2C. As shown in FIGS. 3A, 3B and 3C, frame 10 comprises an upper bore hole 11 and a lower bore hole 13 which may be utilized to secure frame 10 to the top plate 50 and bottom plate 36, respectively. Frame 10 also includes an upper aperture 15a and a lower aperture 15b, both unthreaded, which are configured to receive, hold and support rotating drive rod 20. Frame 10 also has a notch 17 adapted to receive and secure the upper end of stabilizing bracket 26 (shown best in FIG. 4D). Preferably, the frame is made of a lightweight metal material, such as aluminum or titanium. Frame 10 may also have a plurality of clear bore holes (not shown in the figures) to reduce its weight and thereby make the microdrive assemblies incorporating the frames lighter and more tolerable for small, conscious and freely-moving animals.

FIGS. 4A-4E provide more detailed views of the carriage 12, drive rod 20, bracket 26, electrical connector 22 and flexible electrical cable 24 incorporated into the electrical microdrive 28 of modular microdrive assembly 100. Referring to FIGS. 4A and 4B, carriage 12 comprises a threaded bore 16, a platform 14 and two flanges 18a and 18b. Referring to FIG. 4C, drive rod 20 comprises a slotted head 21a and threaded shaft 21b. Threaded bore 16 of carriage 12 is configured to smoothly thread onto threaded shaft 21b, so that the inner threads of threaded bore 16 will come into complementary contact with the outer threads of threaded shaft 21b, and so that rotating drive rod 20 (while it is secured to the frame 10) in either direction around its primary axis will cause carriage 12 to move up and down the drive rod 20 along that primary axis. Preferably, but not necessarily, carriage 12, drive rod 20 and bracket 26 are constructed from the same lightweight metal material used to manufacture frame 10. Electrical connector 22 may comprise any connector capable of being used to electrically couple two or more single- or multi-channel electrical conduits. Electrical connectors suitable for these purposes may be obtained, for example, from Omnetics Connector Corporation, of Minneapolis, Minn. (www.omnetics.com).

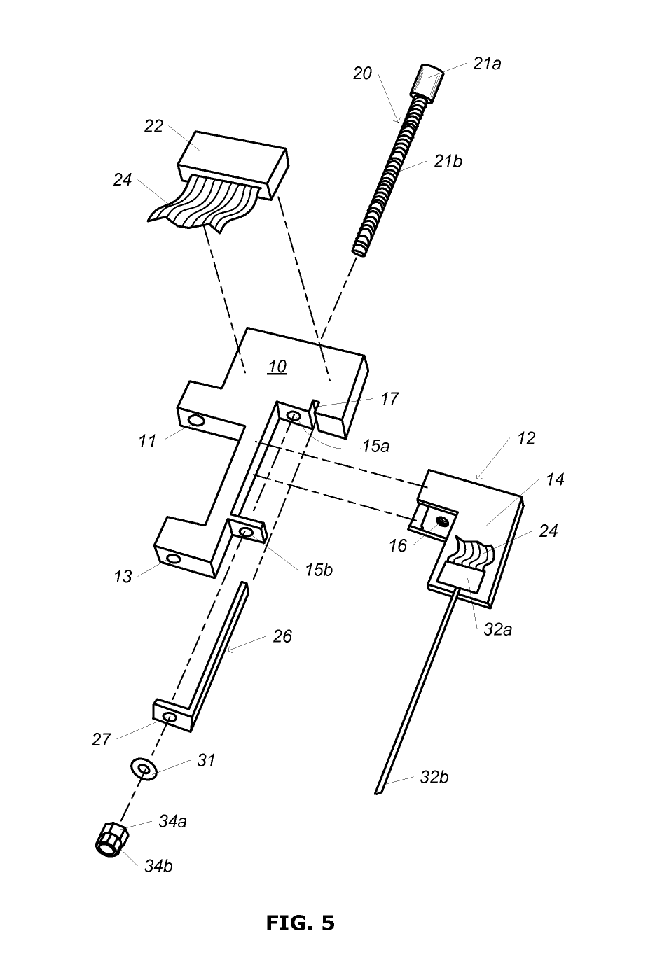

FIG. 5 shows an exploded diagram illustrating how an electrical instrument, such as a silicon probe (designated with reference numerals 32a and 32b), carriage 12, drive rod 20, bracket 26, electrical connector 22, flexible electrical cable 24, washer 31, and nuts 34a and 34b may be assembled in order to produce the exemplary electrical microdrive 28 shown in FIGS. 2A, 2B and 2C. As shown in FIG. 5, the lower end of drive rod 20 is guided through upper support aperture 15a in frame 10 and then threaded through the threaded bore 16 of carriage 12 so that the outer threads of threaded shaft 21b come into and stay in complementary contact with the inner threads of threaded bore 16 on carriage 12. The lower end of drive rod 20 then passes through lower support aperture 15b of frame 10 and the hole 27 at the lower end of bracket 26. The upper end of stabilizing bracket 26 is lodged into notch 17 of frame 10. Drive rod 20 is then secured to bracket 26 and frame 10 by washer 31 and nuts 34a and 34b. It should be appreciated, however, that any one of a variety of different types of fasteners and fastening techniques may be used in order to secure drive rod 20 to frame 10, including for example, a single nut, a c-clip or a pin.

Electrical connector 22 is mounted to the upper portion of frame 10 using any reliable adhesive. Flexible electrical cable 24 is electrically coupled at one end to electrical connector 22, and electrically coupled at its other end to the upper portion 32a of electrical instrument 32a-b, which is mounted to platform 14 of carriage 12. Thus, electrical connector 22 serves to secure the upper end of flexible electric cable 24 to the frame 10, which imparts modularity to the device. This modularity permits an operator, for example, to switch from one external interface cable to another without disturbing the flexible electric cable 24 or the instrument 32a-b. For clarity and ease of understanding, the middle portion of the flexible electrical cable 24 has been removed from the diagram shown in FIG. 5. Typically, flexible electrical cable 24 comprises a ribbon cable, which provides a plurality of separate electrical wires that are electrically coupled to a corresponding plurality of recording or stimulating channels provided by the electrical instrument 32a-b mounted to platform 14 of carriage 12. Multi-channel electrical probes suitable for these purposes may be obtained, for example, from Neuro Probe Technologies, LLC, of Ann Arbor, Mich., USA.

The electrical instrument 32a-b may be made from one or more of a variety of different materials, including, for example, silicon, carbon fiber, glass, metal, plastic or rubber. Platform 14 comprises a surface that is adapted to allow fixedly mounting the instrument to the carriage 12 using, for example, an epoxy adhesive, acrylic, polyurethane, cyanoacrylate, or any other type of reliable adhesive. The electrical instrument 32a-b may also be mounted to the platform 14 on the carriage using a clamp, screw or clip (not shown).

Substantially rigid instruments include instruments having sufficient internal strength and rigidity (stiffness) so that imposing vertical motion (i.e., motion parallel to the Y-axis in FIG. 2C) onto the upper portion 32a of the instrument by moving the carriage 12 and the platform 14 toward the surface of the tissue causes the tip 32b of the instrument to move a substantially equal distance into and through the tissue, and toward a target area, without bending, twisting or turning enough to deflect the tip 32b from its intended path to the target area, even though the instrument 32a-b is not encased in, guided by, or extended through any guiding tubes, hoses or cannulae. In other words, unlike a flexible instrument, such as a wire electrode, a substantially rigid instrument has sufficient internal strength to substantially avoid significant changes in its geometry due to the external compression and shearing forces caused by applying pressure to the upper portion 32a of the instrument in an effort to force the tip 32b of the instrument to penetrate and move through the tissue toward the target area. While some deformation of even substantially rigid instruments is possible and permissible, it tends to be very minimal and does not significantly effect the direction of movement and positioning of the tip of the instrument. Therefore, it is not necessary to extend the substantially rigid instruments through tubular guides, hoses or cannulae to avoid deflections from the target caused by geometric deformations imposed by compression or shearing forces arising from movement of the tip through the tissue.

Assembling the various components in the manner described above and as shown by the exploded diagram of FIG. 5 produces the electrical microdrive 28 depicted in FIGS. 2A, 2B and 2C. Returning to FIGS. 2A and 2B, it may be seen that, due to the complementary contact 30 between the outer threads of threaded shaft 21b on drive rod 20 and the inner threads of threaded bore 16 on carriage 12, rotating slotted head 21a in one direction will necessarily urge the entire carriage 12 to move toward the lower end of drive rod 20, thereby forcing the electrical instrument 32a-b mounted to platform 14 to move to a lower position, which causes the instrument to penetrate (or further penetrate) any tissue located immediately below the instrument 32a-b. Similarly, rotating slotted head 21a in the opposite direction will necessarily urge carriage 12 to move away from the lower end of drive rod 20, thereby forcing the electrical instrument 32a-b mounted to platform 14 to move to a higher position, which retracts or extracts the instrument from tissue located immediately below it. A reasonably effective rate of travel for electrical instrument 32a-b is about 320 microns per single revolution of slotted head 21a. However, larger or smaller incremental movement by electrical instrument 32a-b may be achieved by varying the size of the helixes forming the threads on threaded shaft 21b and threaded bore 16.

It should also be apparent from FIGS. 2A, 2B and 2C that assembling electrical microdrive 28 in the manner described above and as shown by the exploded diagram of FIG. 5 brings carriage 12 into slidable contact with frame 10 in such a way that flanges 18a and 18b on carriage 12 each wrap part-way around opposite lateral sides of frame 10. In this position, the contact between frame 10 and flanges 18a and 18b inhibit the carriage 12 from tilting or rotating around the Y-axis while the drive rod 20 is being rotated (see FIG. 2C).

Depending on a number factors, including, for example, the diameter of instrument being inserted, the density and resistance of the penetrated tissue, and obstacles that may be located in the path of the instrument, the inventors of the present invention have observed that operating electrical microdrive 28 to push or pull the tip of electrical instrument 32a-b further into or out of the subject tissue can produce relatively significant insertion and extraction forces at 32b that attempt to tilt or rotate the carriage 12 about tilt axes that are substantially transverse to the intended rotation of drive rod 20 (i.e., around the X- and Z-axes shown in FIG. 2C). If carriage 12 is allowed to tilt or rotate in these directions, then electrical instrument 32a-b may be deflected away from its intended target area, and the threaded bore 16 of carriage 12 may exert sufficient sideways forces at complementary contact 30 to bend, torque, warp or deflect drive rod 20, or otherwise move drive rod 20 out of its intended and correct alignment relative to the frame 10. Accordingly, one purpose of bracket 26, which is installed according to the diagram shown in FIG. 5 so that it comes into solid slidable contact with carriage 12, is to inhibit carriage 12 from rotating about the X- or Z-axes. Bracket 26 also serves to hold steady the lower end of drive rod 20 while it is being rotated, thereby preventing drive rod 20 from coming out of alignment due to the insertion and extraction forces produced by the normal operation of electrical microdrive 28. Notably, bracket 26 also operates, in conjunction with flanges 18a and 18b, to reduce the possibility that carriage 12 may rotate about the Y-axis.

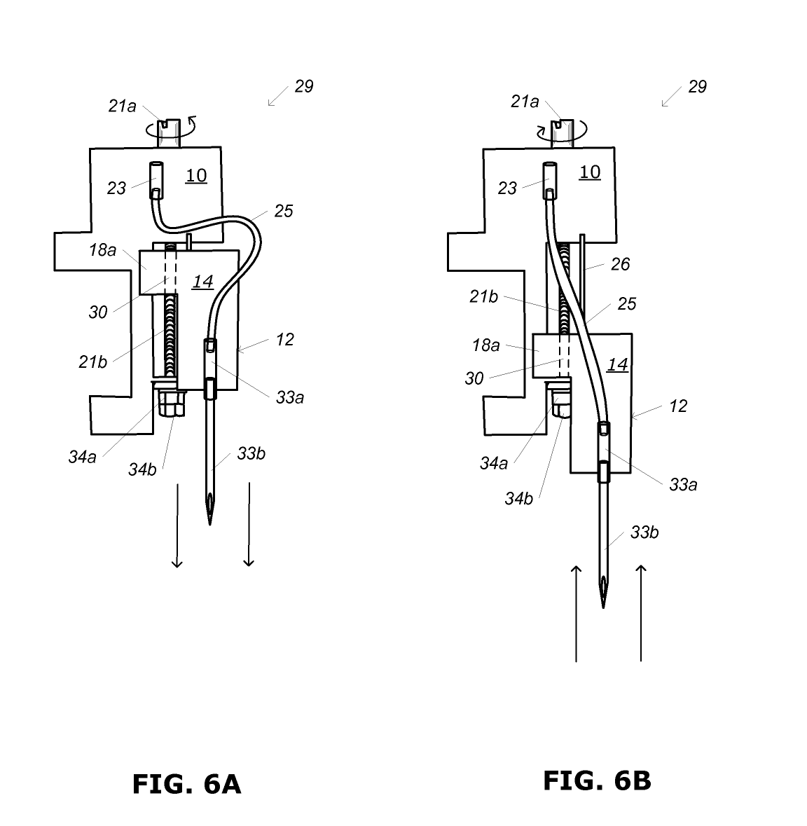

FIGS. 6A and 6B show in the retracted and extended positions, respectively, a fluid microdrive 29 suitable for positioning a fluid-transporting device, such as a needle, according to an alternative embodiment of the invention. As shown in FIGS. 6A and 6B, the configuration of a fluid microdrive 29 is substantially the same as the configuration of the electrical microdrive 28 (shown best in FIGS. 2A, 2B and 2C), except that electrical connector 22 is replaced by a fluid connector 23, and the flexible electrical cable 24 is replaced by a flexible tube 25. Instead of mounting an electrical instrument 32a-b to platform 14 of carriage 12, a fluid instrument 33a-b is mounted to platform 14. Fluid tube 25 is coupled at one end to the fluid-transporting instrument 33a-b mounted to the platform 14 of carriage 12. Fluid tube 25 is connected at its other end to the fluid connector 23, which, like electrical connector 22, is mounted to an upper region of the frame 10. The fluid connector 23 may comprise any structure, e.g., a short tube, a bore hole, a straw, etc., capable of securing the top of fluid connector 25 to the frame 10 and providing a channel through which fluids may pass as it moves from the fluid tube 25 to an external interface tube (not shown), or vice versa. Alternatively, fluid connector 23 may be integrated into the frame 10.

Fluid microdrive 29 operates substantially in the same manner as electrical microdrive 28. When slotted head 21a is rotated in the counterclockwise direction, for example, the complementary contact 30 between the outside threads of threaded shaft 21b and the inner threads of threaded bore 16 on carriage 12 urges carriage 12 toward the lower end of the drive rod 20, thereby lowering platform 14, which causes the needle (fluid instrument 33a-b) to move toward or further into any tissue immediately below it. Conversely, when slotted head 21a is rotated in the clockwise direction, the complementary contact 30 between the outside threads of threaded shaft 21b and the inner threads of threaded bore 16 on carriage 12 urges carriage 12 away from the lower end of drive rod 20, thereby raising platform 14, which causes the needle to move away from or out of any tissue immediately below it.

Although not specifically shown in the figures, it should be understood and appreciated that a plurality of fluid microdrives 29 (as shown in FIGS. 6A and 6B) may be attached to a top plate, a bottom plate and a protective cover, as described above with respect to the electrical microdrives 28, in order to produce a modular microdrive assembly for fluid-transporting instruments that will be very similar to the microdrive assembly for electrical instruments depicted in FIG. 1. However, when the microdrive assembly is for use with fluid-transporting devices, the fluid connector 23 mounted to the frame 10 will be fluidly coupled to one end of a fluid interface tube (not shown), such as a rubber or plastic tube (instead of an electrical interface, such as interface cable 70 in FIG. 1). The fluid interface tube is then connected to a remote fluid reservoir configured to hold fluids that are collected from or injected into the body, a fluid pumping mechanism, or both.

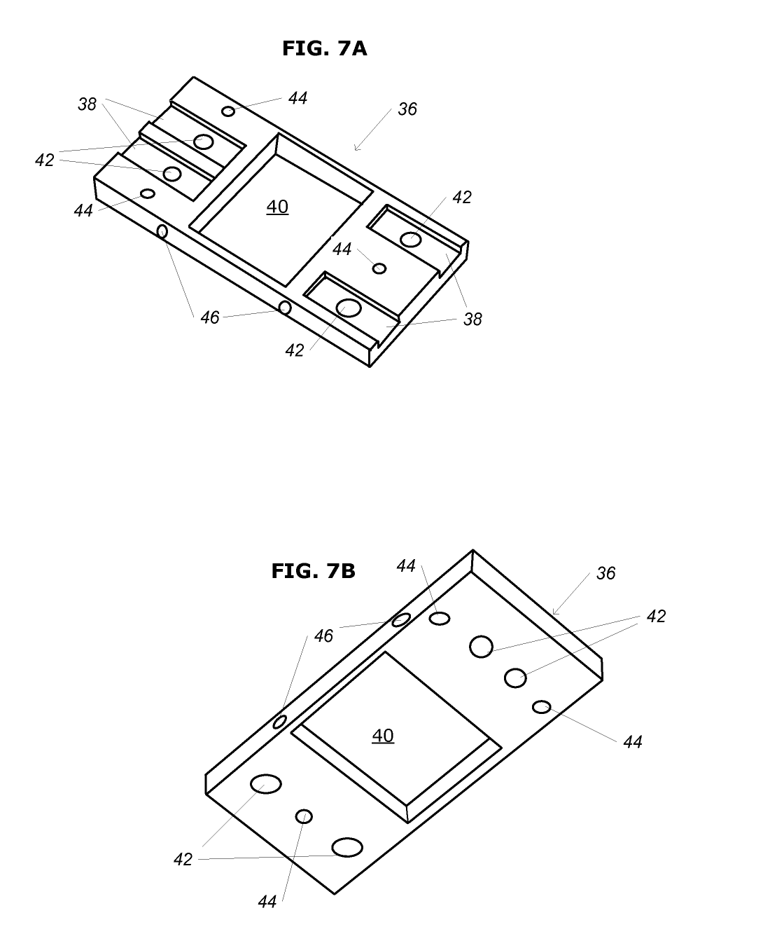

FIGS. 7A and 7B show, respectively, a top perspective view and a bottom perspective view of a bottom plate 36 configured to accommodate a plurality of microdrives (in this case, four microdrives), such as the electrical microdrive 28 and the fluid microdrive 29, described above. As shown best in FIG. 7A, bottom plate 36 comprises a plurality of microdrive slots 38, a plurality of microdrive slot holes 42, a plurality of anchor screw holes 44, a plurality of pinholes 46 and a bottom void 40. Anchor screw holes 44 are configured to accept slope-headed anchoring screws (indicated in FIG. 1 by reference numeral 59), which are inverted and threaded into the bottom side of bottom plate 36. When the bottom plate 36 is attached to an animal's skull using dental cement, the inverted slope-headed anchoring screws 59 are immersed in dental cement and thereby assist in holding the bottom plate, and consequently, the entire modular microdrive assembly 100, securely to the animal's skull. Metal pins (not shown in the figures) may be inserted into pinholes 46 and bent downward toward the skull to provide additional structure that can be cemented or screwed to the skull in order to provide additional stability.

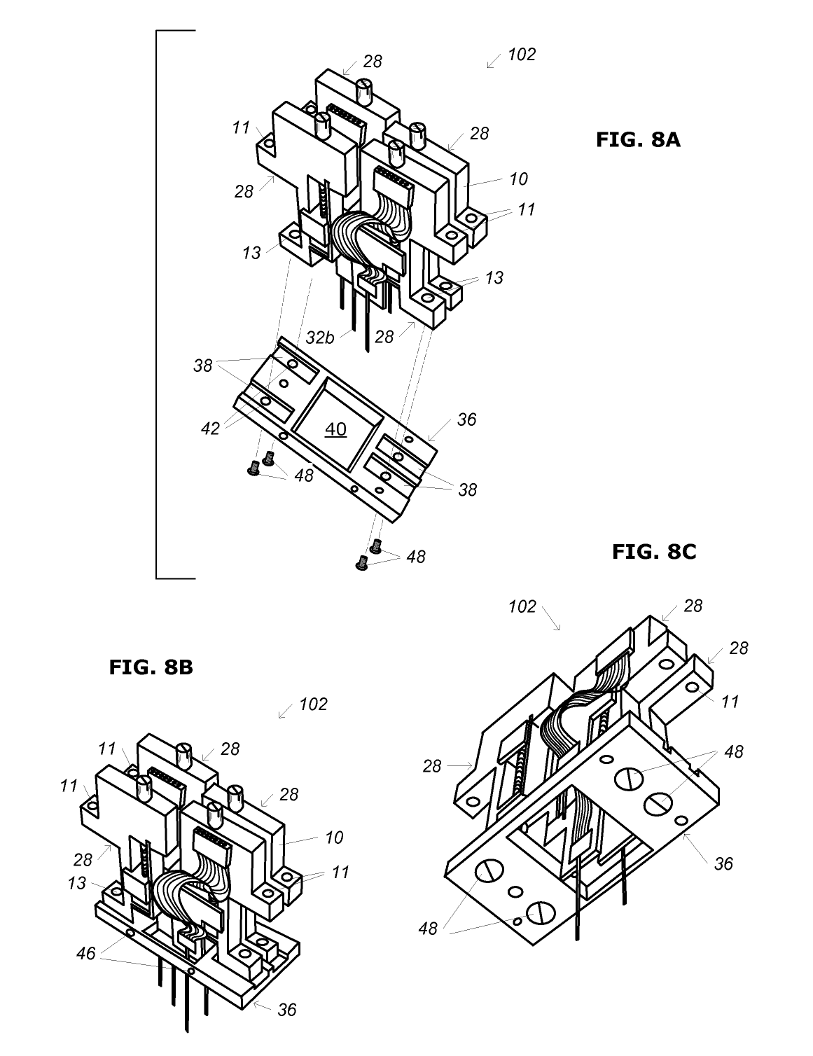

FIGS. 8A, 8B and 8C show left perspective views (from above and below) of a modular microdrive assembly (generally designated 102) according to another embodiment of the invention. As shown in FIG. 8A, the feet of four electrical microdrives 28 are slotted into four microdrive slots 38 on the top of bottom plate 36 so that the tips of four electrical instruments 32b mounted to four frames 10 may move freely through bottom void 40 when the drive rods in the microdrives 28 are rotated. Preferably, the feet of the four microdrives 28 are removably secured to bottom plate 36 by inserting four threaded securing screws 48 through the slot holes 42 and threading them into threaded lower bore holes 13 of frames 10. Notably, however, the electrical microdrives 28 may also be permanently secured to the bottom plate 36 using, for example, glue, cement or solder joints. FIGS. 8B and 8C show the assembled modular microdrive assembly 102 after the bottom plate 36 has been attached. Bottom plate 36 holds the assembly to the subject animal's body and serves to orient the electrical microdrives 28 (and therefore the penetrating instruments mounted to the microdrives) to the target.

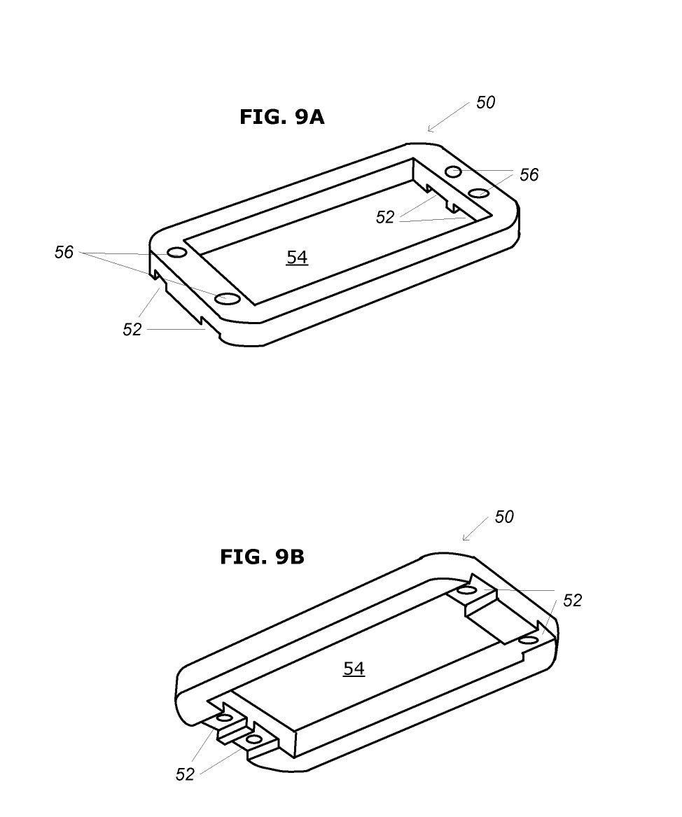

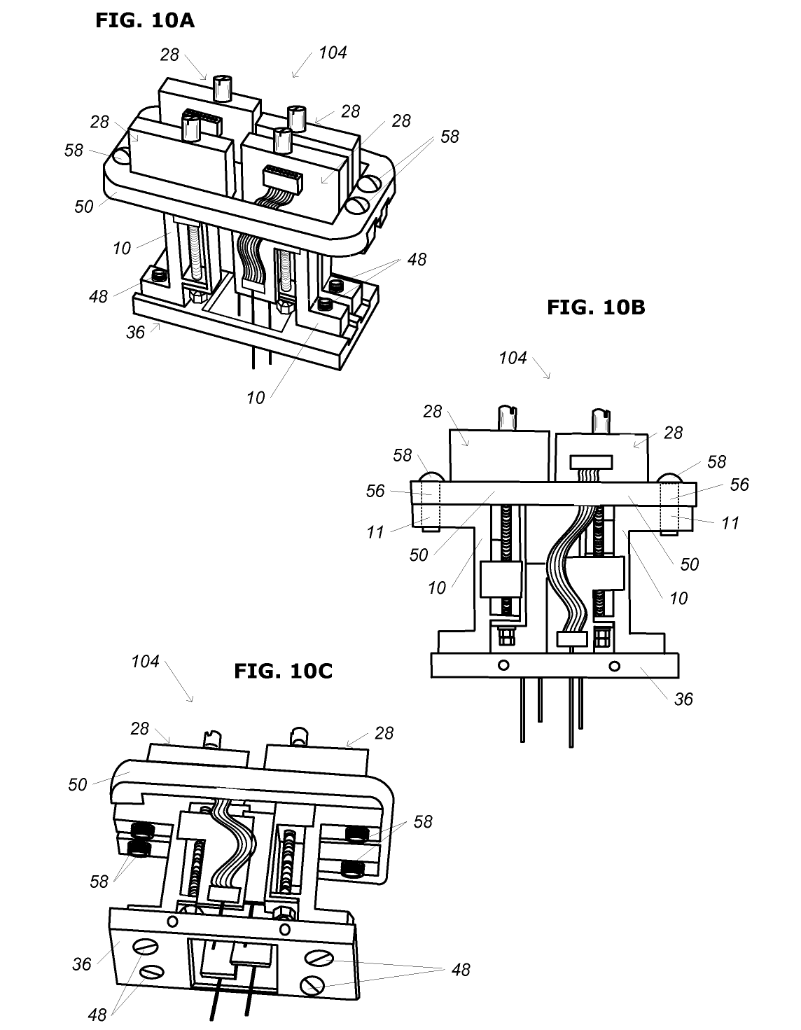

FIGS. 9A and 9B show, respectively, a top perspective view and a bottom perspective view of the top plate 50 for the microdrive assembly. As shown in FIGS. 9A and 9B, top plate 50 comprises a top void 54, through which the upper portions of microdrives will pass, and a plurality of microdrive slots 52 adapted to receive and hold in place certain parts of a plurality of microdrives. FIGS. 10A, 10B and 10C show, respectively, a left perspective view (from above), a left side orthogonal view and a bottom perspective view of a modular microdrive assembly (generally designated 104) with both the top and bottom plates installed. As shown in FIGS. 10A, 10B and 10C, the plurality of microdrives 28 are removably secured to the top plate 50 by passing a plurality of threaded securing screws 58 through a plurality of slot holes 56 in top plate 50, and then threading those securing screws 58 into the upper bore holes 11 drilled into the frames 10. However, the microdrives 28 may also be permanently secured to the top plate 50 using, for example, glue, cement or solder joints. The top plate 50 provides additional stability for the plurality of electrical microdrives 28 secured to bottom plate 36, and also provides a stable anchor for a protective cover for the microdrive assembly.

FIGS. 11A, 11B and 11C show, respectively, a left perspective view (from above), a rear perspective view (from above) and a left perspective view (from below) of an exemplary protective cover for a modular microdrive assembly, such as modular microdrive assembly 104 shown in FIGS. 10A, 10B and 10C. The exemplary protective cover 60 comprises a substantially rectangular-shaped and partially-enclosed receptacle 61 having a hollow interior chamber that is substantially open to access on its bottom-facing side. Two rocker clips 62 are attached to opposite sides of the receptacle 61. The lower ends of the rocker clips 62 are configured to automatically spread apart and then spring back and firmly grip the underside of the top plate 50 when pressure is applied to the top or sides of the protective cover 60 in order to force the lower ends of the rocker clips 62 and the open side of receptacle 61 down over the top of modular microdrive assembly 104. The rocker clips 62 are biased to engage and grip the bottom side of top plate 50 with sufficient gripping force to firmly hold protective cover 60 to the microdrive assembly 104 while the microdrive assembly remains surgically attached to conscious and behaving laboratory animals. Pressing the upper ends of the rocker clips 62 toward the receptacle 61 spreads apart the lower ends of the rocker clips 62, thereby releasing the protective cover 60 from the top plate 50 and modular microdrive assembly 104.

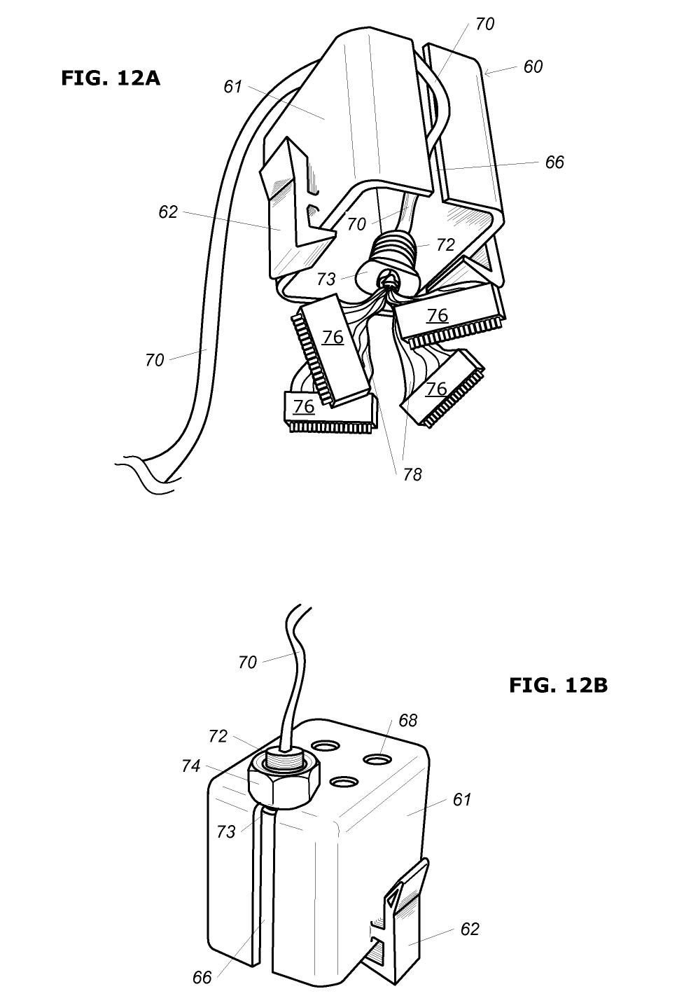

Protective cover 60 also includes on its top and front sides, respectively, an outlet 64 that intersects a slit 66, which together are configured to permit passing the wires of an interface cable (discussed in more detail below with reference to FIG. 12) from the inside of the hollow interior chamber of receptacle 61 to the outside of the protective cover 60. The top of the protective cover 60 also contains a plurality of drive rod access holes 68, which permit access to and rotation of the drive rods 20 on the microdrives 28 without removing the protective cover 60 from the modular microdrive assembly 104.

The protective cover 60 may be made from any suitably rigid and/or resilient material, such as plastic, metal or glass, to provide protection for the drive rods, electrical connectors, fluid connectors and interface wires situated within the interior hollow chamber of receptacle 61 against intentional and inadvertent external forces and influences produced, for example, by the subject animal or other animals in the immediate vicinity. Depending on the geometry of the modular microdrive assembly it is intended to cover and protect, protective cover 60 may have any one of a variety of different shapes, including without limitation, a cube, a dome, a pyramid or a cylinder. The protective cover 60 may also be transparent, translucent or opaque.

FIGS. 12A and 12B show, respectively, a left perspective view (from below) and a right perspective view (from above) of the protective cover 60 fitted to the interface cable 70 and various interface components. As shown best in FIG. 12A, interface cable 70, which may be electrically coupled, for example, to a remote signal processor or remote signal generator (not shown) passes through slit 66 and outlet 64 into the interior hollow chamber of receptacle 61 of protective cover 60, where it is electrically coupled to an interface coupling screw 72. A plurality of interface wires 78 pass from the interface cable 70, through the interface coupling screw 72 and into a plurality of interface electrical connectors 76. The plurality of interface electrical connectors 76 are configured to mate with and electrically couple to a corresponding plurality of electrical connectors 22 mounted to the plurality of electrical microdrives 28. As stated above, electrical connectors suitable for use as electrical connectors 22 and interface electrical connectors 76 may be obtained, for example, from Omnetics Connector Corporation, of Minneapolis, Minn. (www.omnetics.com).

To secure the interface cable 70 to the protective cover 60, the top portion of interface coupling screw 72 is pushed through outlet 64. The lower portion of interface coupling screw 72 has a circular flange 73 having a sufficient diameter to prevent interface coupling screw 72 from passing entirely through outlet 64 to the outside of protective cover 60. A nut 74 is threaded over the top of interface coupling screw 72, which firmly holds interface coupling screw 72 to the top of the interior hollow chamber of receptacle 61.

Because interface cable 70 is firmly attached to the protective cover 60 by interface coupling screw 72 and nut 74, forces exerted against interface cable 70 (such as tugging and pulling by the subject animal) are transmitted to and dissipated by the rigid and stable dispositions of the top and bottom plates, which are themselves tightly secured to the subject animal's skull with the dental cement. This arrangement creates less strain on the individual interface wires 78, interface electrical connectors 76 and electrical connectors 22 situated inside the protective cover 60. To remove the interface cable 70 from the protective cover 60, nut 74 is removed from interface coupling screw 72 so that interface coupling screw 72 and a sufficient length of interface cable 70 may be drawn into and through the interior hollow chamber of receptacle 61, so that interface cable 70 can pass through the slit 66 and be entirely removed from protective cover 60.

As shown best in FIG. 12A, a portion of the circular outside edge of flange 73 is flattened so that contact with the front interior wall of receptacle 61 will not prevent the upper portion of interface coupling screw 72 from fitting into outlet 64. Alternatively, protective cover 60 may be arranged so that outlet 64 is positioned further away from the interior walls of receptacle 61 (e.g., in the center of the top side of protective cover 60), thereby reducing the possibility that flange 73 will ever come into contact with any interior side walls of receptacle 61 when the upper portion of interface coupling screw 72 is pushed into outlet 64.

Other microdrive assembly configurations, besides the rectangular-shaped configurations described above, are possible. For example, although the modular microdrive assemblies 100, 102 and 104, described above, are configured so that four microdrives are held in place by rectangular-shaped top and bottom plates, one may also manufacture, for instance, larger or smaller top and bottom plates to accommodate a larger or smaller number of microdrives. Thus, it may be possible, depending on the size and geometry of the implantation site, to put six, eight, ten or more microdrives in a sufficiently-large set of top and bottom plates. In addition, annular-shaped top and bottom plates may be manufactured, which could then provide support for placing a multiplicity of microdrives in a circular configuration. In such configurations, the number of microdrives that could be attached to the top and bottom plates is only limited by the size and thickness of the microdrives, along with the size and geometry of the implantation site. Moreover, it should be appreciated that the frames, as well as the top and bottom plates, do not have to have the same, or even similar, shapes. Thus, a variety of differently-shaped frames, top plates and bottom plates may be combined to produce a single modular microdrive assembly, and to facilitate placing a plurality of microdrives on the single microdrive assembly in a plurality of different orientations to accommodate a variety of different and independent insertion angles for the instruments, as well as simultaneously attaching and independently positioning a mix of different types of instruments.

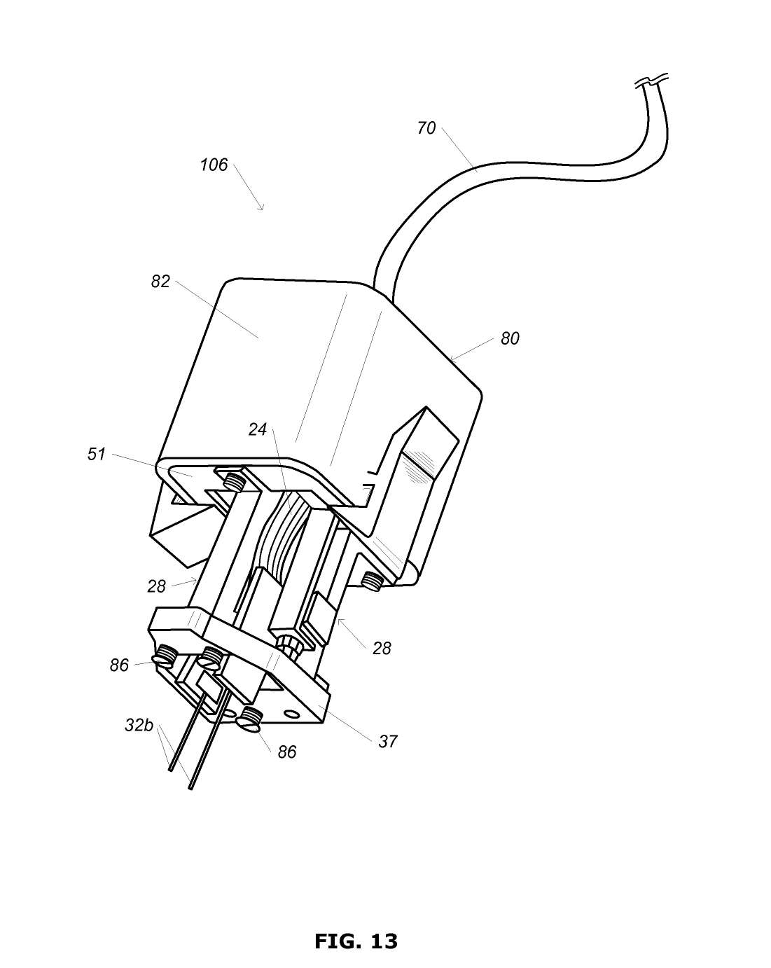

FIG. 13 shows, for example, a modular microdrive assembly 106 according to an alternative embodiment of the invention, wherein the bottom plate 37 is substantially triangular, while the top plate 51 is substantially square. Notably, the modular microdrive assembly 106 of FIG. 13 is configured to hold only two electrical microdrives 28 and position only two electrical instruments 32a-b. Tapering the top and/or bottom plates may change the footprint of the microdrive assembly so that it better fits the head (or other body part) of the subject animal, and also may provide a lighter and less bulky microdrive assembly for conscious and freely-moving animals to tolerate. The inventors of the present invention have found, for example, that bottom plates tapered at one end like the bottom plate shown in FIG. 13 are typically more effective and easier to implant when the modular microdrive assembly is implanted on the head of a mouse. A plurality of slope-headed anchoring screws 86 may be inserted into the underside of triangular-shaped bottom plate 37 to secure the microdrive assembly 106 to the mouse's head.

The size and shape of the top plate may determine the size and shape of the protective cover. For instance, unlike the substantially rectangular-shaped protective cover 60, described above with reference to FIGS. 11A, 11B and 11C, the protective cover 80 for microdrive assembly 106 in FIG. 13 is substantially cubic in shape.

In general, using an embodiment of the present invention to record signals produced in the body of an animal comprises the steps of: (1) mounting a recording instrument, such as an electrode, on the carriage of the microdrive; (2) coupling one end of the flexible electrical cable to the instrument; (3) coupling the other end of the flexible cable to the electrical connector mounted on the microdrive; (4) securing the microdrive to the bottom plate; (5) securing the top plate to the microdrive; (6) attaching the bottom plate to the body so that the bottom void in the bottom plate is adjacent to the location of the body where the instrument is to be inserted; (7) electrically coupling one end of an interface cable to the electrical connector mounted to the microdrive; (8) passing at least a portion of the interface cable through the protective cover; (9) connecting the interface cable to a remote signal processor; (10) attaching the protective cover to the top plate; and (11) rotating the drive rod in one direction to produce a force at the complementary contact that urges the carriage closer to the location, thereby pushing the instrument mounted on the carriage through the bottom void and into the body. The steps do not necessarily have to be performed in this order.

Although the exemplary embodiments, uses and advantages of the invention have been disclosed above with a certain degree of particularity, it will be apparent to those skilled in the art upon consideration of this specification and practice of the invention as disclosed herein that alterations and modifications can be made without departing from the spirit or the scope of the invention, which are intended to be limited only by the following claims and equivalents thereof. It can be appreciated, for example, that the concepts and general procedures, as described above with reference to particular embodiments, are valid for positioning instruments in any animal (including humans), and are not limited to use with the aforementioned laboratory animals.

What is claimed is:

1. A microdrive for positioning the tip of a substantially rigid instrument in the body of an animal, comprising:
a frame;
a bottom plate having a bottom void therein, said bottom plate being adapted to be fixedly secured to the frame and surgically attached to the body so that the bottom void is fixedly disposed about a location on the surface of the body where the tip is to be inserted;
a carriage having a threaded bore and a platform for fixedly holding the substantially rigid instrument, said platform being configured to hold the substantially rigid instrument so as to permit the tip to extend through the bottom void toward the surface of the body without passing the substantially rigid instrument through a tubular guide; and
a drive rod rotatably mounted to the frame, the drive rod having an upper end, a lower end and a threaded shaft therebetween, the threaded shaft passing through the threaded bore on the carriage so that the threads of the threaded shaft are in complementary contact with the threads of the threaded bore;
whereby rotating the drive rod in one direction produces a force at the complementary contact which urges the platform on the carriage closer to the surface, thereby forcing the tip of the substantially rigid instrument to penetrate the body.

2. The microdrive of claim 1, whereby rotating the drive rod in the opposite direction produces an opposite force at the complementary contact which urges the platform on the carriage away from the surface, thereby pulling the substantially rigid instrument through the bottom void and extracting the tip from the body.

3. The microdrive of claim 1, wherein the substantially rigid instrument comprises an electrode.

4. The microdrive of claim 1, wherein the substantially rigid instrument comprises a mechanical probe.

5. The microdrive of claim 1, wherein the substantially rigid instrument comprises a needle.

6. The microdrive of claim 1, further comprising:
an electrical connector mounted to the frame; and
a flexible electrical cable electrically coupled at one end to the substantially rigid instrument and electrically coupled at the other end to the electrical connector;
whereby electrical signals recorded by the substantially rigid instrument are transmitted to the electrical connector via the flexible electrical cable.

7. The microdrive of claim 6, wherein electrical signals introduced to the electrical connector are transmitted to the substantially rigid instrument via the flexible electrical cable.

8. The microdrive of claim 1, further comprising:
a fluid connector mounted to the frame; and
a flexible tube in fluid communication at one end with the substantially rigid instrument and in fluid communication at the other end with the fluid connector;
whereby fluid extracted from the body through the substantially rigid instrument is carried to the fluid connector via the flexible tube.

9. The microdrive of claim 8, wherein fluid introduced to the fluid connector is carried to the substantially rigid instrument by the flexible tube.

10. The microdrive of claim 1, wherein the carriage comprises a flange in slidable contact with the frame, thereby inhibiting the carriage from rotating about an axis parallel to the rotational axis of the drive rod.

11. The microdrive of claim 1, further comprising a bracket, coupled to the frame and in slidable contact with the carriage, thereby inhibiting the carriage from rotating about an axis substantially transverse to the rotational axis of the drive rod.

12. The microdrive of claim 1, wherein the upper end of the drive rod comprises at least one of the following:
a turnable knob,
a crank,
a thumbwheel,
a bolt head,
a slotted head,
a headless slot,
a socketed head,
a headless socket,
a Phillips head,
a square-drive head,
a Torx head,
a Tri-Wing head,
a Torq-Set head, or
a spanner head.

13. The microdrive of claim 1, further comprising a motor drive, coupled to the drive rod, configured to automatically rotate the drive rod by a specified amount.

14. The microdrive of claim 1, further comprising a fastener, coupled to the lower end of the drive rod, which secures the lower end of the drive rod to the frame without impeding the rotational movement of the drive rod, said fastener comprising at least one of:
a washer,
a nut,
a c-clip, or
a pin.

15. A modular microdrive assembly for positioning the tips of substantially rigid instruments in the body of an animal, comprising:
a plurality of microdrives, each comprising a frame, a carriage having a threaded bore and a platform, and a drive rod rotatably mounted to said frame, the drive rod comprising an upper end, a lower end and a threaded shaft therebetween, the threaded shaft passing through the threaded bore so that the threads of the threaded shaft are in complementary contact with the threads of the threaded bore;
a bottom plate having a bottom void therein, said bottom plate being configured to be fixedly secured to the plurality of microdrives and surgically attached to the body so that the bottom void is fixedly disposed about a location on the surface of the body where the tips are to be inserted;
a plurality of substantially rigid instruments mounted on the plurality of platforms on the plurality of carriages, respectively, each platform being configured to hold each substantially rigid instrument so as to permit the tip of said each substantially rigid instrument to be extended through the bottom void toward the surface of the body without passing each substantially rigid instrument through a tubular guide; and
whereby rotating the drive rods for the plurality of microdrives in one direction produces a respective plurality of forces at the respective plurality of complementary contacts that urge the plurality of platforms on the plurality of carriages closer to the surface, thereby pushing the plurality of substantially rigid instruments through the bottom void and advancing the tips into the body.

16. The modular microdrive assembly of claim 15, wherein independently rotating the drive rods for the plurality of microdrives in one direction produces a respective plurality of independent forces at the respective plurality of complementary contacts that independently urge the carriages toward the surface, thereby independently pushing the plurality of substantially rigid instruments through the bottom void and independently advancing the tips into the body.

17. The modular microdrive assembly of claim 15, wherein rotating the drive rods of the plurality of microdrives in the opposite direction produces a respective plurality of opposite forces at the respective complementary contacts which urge the carriages away from the surface, thereby pulling the plurality of substantially rigid instruments through the bottom void and withdrawing the tips out of the body.

18. The modular microdrive assembly of claim 17, wherein independently rotating the drive rods of the plurality of microdrives in the opposite direction produces a respective plurality of independent opposite forces at the respective complementary contacts which independently urge the carriages away from the surface, thereby independently pulling the plurality of substantially rigid instruments through the bottom void and independently withdrawing the tips out of the body.

19. The modular microdrive assembly of claim 15, wherein said plurality of substantially rigid instruments comprises a plurality of electrodes.

20. The modular microdrive assembly of claim 15, wherein said plurality of substantially rigid instruments comprises a plurality of mechanical probes.

21. The modular microdrive assembly of claim 15, wherein said plurality of substantially rigid instruments comprises a plurality of needles.

22. The modular microdrive assembly of claim 15, further comprising a top plate removably secured to the plurality of microdrives, thereby limiting lateral, vertical and rotational movement of the plurality of microdrives relative to the bottom plate.

23. The modular microdrive assembly of claim 15, further comprising:
a plurality of electrical connectors mounted respectively to the plurality of microdrives;
a plurality of flexible electrical cables, each electrically coupled at one end to a substantially rigid instrument mounted on the platform of a carriage of a microdrive and each electrically coupled at the other end to one of said plurality of electrical connectors; and
an interface to a remote signal processor, said interface being electrically coupled to the plurality of electrical connectors;
whereby signals recorded by the instruments are transmitted to the remote signal processor via the plurality of flexible electrical cables, the plurality of electrical connectors and the interface.

24. The modular microdrive assembly of claim 23, further comprising a protective cover that at least partially encloses at least a portion of said interface, thereby protecting said at least a portion against external forces.

25. The modular microdrive assembly of claim 24, wherein the protective cover comprises a substantially-rigid cap having a hollow interior chamber into which said portion passes.

26. The modular microdrive assembly of claim 15, further comprising a plurality of anchoring screws which are inserted into the underside of the bottom plate so that the heads of the anchoring screws are adjacent to the body when the bottom plate is surgically attached.

27. A method for positioning the tip of a substantially rigid instrument in the body of an animal using a microdrive, the microdrive comprising a frame, a bottom plate having a bottom void therein, a carriage having a threaded bore and a platform, and a drive rod rotatably mounted to the frame, the drive rod having an upper end, a lower end and a threaded shaft therebetween, the threaded shaft passing through the threaded bore on the carriage so that the threads of the threaded shaft are in complementary contact with the threads of the threaded bore, said method comprising:
fixedly mounting the substantially rigid instrument to the platform on the carriage;
surgically attaching the bottom plate to the body so that the bottom void is fixedly disposed about a location on the surface of the body where the tip is to be inserted;
fixedly securing the frame to the bottom plate so that the tip of the substantially rigid instrument mounted on the platform may be extended through the bottom void toward the surface of the body without passing the substantially rigid instrument through a tubular guide; and
rotating the drive rod in one direction to produce a force at the complementary contact which urges the platform on the carriage closer to the surface, thereby forcing the tip of the substantially rigid instrument to penetrate the body.

28. The method of claim 27, further comprising rotating the drive rod in the opposite direction to produce an opposite force at the complementary contact that urges the platform on the carriage away from the surface, thereby pulling the substantially rigid instrument through the bottom void and extracting the tip from the body.

29. The method of claim 27, wherein the substantially rigid instrument comprises an electrode.

30. The method of claim 27, wherein the substantially rigid instrument comprises a mechanical probe.

31. The method of claim 27, wherein the substantially rigid instrument comprises a needle.

32. The method of claim 27, further comprising:
mounting an electrical connector to the frame;
electrically coupling one end of a flexible electrical cable to the substantially rigid instrument;
electrically coupling the other end of the flexible electrical cable to the electrical connector; and
transmitting electrical signals recorded by the substantially rigid instrument to the electrical connector via the flexible electrical cable.

33. The method of claim 32, further comprising transmitting electrical signals introduced to the electrical connector to the substantially rigid instrument via the flexible electrical cable.

34. The method of claim 27, further comprising:
mounting a fluid connector to the frame;
fluidly coupling one end of a flexible tube to the substantially rigid instrument;
fluidly coupling the other end of the flexible tube to the fluid connector; and
transporting fluid extracted from the body through the substantially rigid instrument to the fluid connector via the flexible tube.

35. The method of claim 34, further comprising using the flexible tube to transport the fluid introduced into the fluid connector to the substantially rigid instrument.

36. The method of claim 27, further comprising:
coupling a motor drive to the drive rod; and
rotating the drive rod a specified amount automatically with the motor drive.
